(19) United States
(12) Patent Application Publication
(10) Pub. No.: US 2004/0133200 A1
(43) Pub. Date: Jul. 8, 2004

(54) Apparatus and method for maintaining bones in a healing position

(76) Inventors: David S. Ruch, Winston-Salem, NC (US); Richard M. Bryant, Clemmons, NC (US)

Correspondence Address: BAKER BOTTS L.L.P., 2001 ROSS AVENUE, SUITE 600, DALLAS, TX, 75201-2980, US

(21) Appl. No.: 10/712,574

(22) Filed: Nov. 13, 2003

Related U.S. Application Data
(60) Provisional application No. 60/426,505, filed on Nov. 15, 2002.

(51) Int. Cl. A61B 17/60

(52) U.S. Cl. 606/54

(57) ABSTRACT

An apparatus is provided to reduce bony fragments and maintain a human's wrist or hand in a healing position. The apparatus may include a first clamp assembly operable to removably mount to an external fixator when the external fixator is coupled to a first bone of a human. A second clamp assembly may be releasably engaged with at least one bone pin embedded in a second bone of a human. A connector rod may be provided to join the first clamp assembly to the second clamp assembly.

### RELATED APPLICATION

This application claims priority to U.S. Provisional Patent Application Serial No. 60/426,505, filed Nov. 15, 2002, and entitled “Ulna Outrigger.”

### TECHNICAL FIELD

The present invention relates generally to an apparatus and method for use in fixation healing and reduction of bones and/or soft tissue and more specifically to an apparatus and method for maintaining a healing position of a human's wrist or hand using external fixation.

### BACKGROUND OF THE INVENTION

The use and advantages of external fixators for the treatment of fractures or other injuries or conditions of the wrist are well known. External fixators are generally used for comminuted unstable distal radius and/or ulna fractures, both with and without internal fixation of the fracture fragments themselves. External fixation is generally indicated for fractures that are comminuted and unstable. This is because unstable fracture fragments tend to displace despite external cast support alone if the bone fragments cannot be held in a stable position.

Current designs of external fixators include both static models, which hold a fracture in a stable position, and dynamic external fixators which stabilize a fracture while allowing for some extremity movement. Dynamic external fixators offer the advantage of maintaining traction at a fracture site while allowing limited wrist motion. This feature is important because limited wrist motion has been shown to improve the longterm functional results after healing of some wrist fractures.

Generally, fixators use the principal of longitudinal traction applied to the skeleton by proximal and distal pins at either end of a bone that is fractured. In the case of the wrist, this may occur across the wrist joint with proximal pins in the radial shaft and distal pins in the metacarpal bones. Alternatively, pins may be placed in the proximal or midshaft radius and the distal radius. A dynamic external fixator applied to the wrist allows the combination of stability of the distal radius fracture while allowing limited freedom of movement of the joint itself. This method allows earlier recovery from the stiffness normally associated with complete immobilization of the fracture while maintaining adequate alignment of the fracture during the process of healing.

### SUMMARY OF THE INVENTION

In accordance with teachings of the present invention, an apparatus and method for reducing bone fragments and maintaining a human's wrist and hand in a healing position are provided. The apparatus may include a first clamp assembly operable to removably mount to an external fixator wherein the external fixator may be coupled to a bone of a human, the first clamp assembly further operable to couple to a connector rod and a second clamp assembly operable to releasably couple to at least one bone pin embedded in a bone of a human and a connector rod operable to join the first clamp assembly to the second clamp assembly.

In another embodiment an apparatus for reducing bone fragments and maintaining a healing position of one or more bones and/or soft tissue of a human's wrist. At least one bone and/or soft tissue of the human's wrist may be fractured or soft tissue may be damaged. The apparatus includes a first clamp assembly operable to removably mount to an external fixator wherein the external fixator is coupled to a radius of a human, the first clamp assembly further operable to couple to a connector rod, a second clamp assembly operable to releasably couple to at least one bone pin embedded in the ulna of a human and a connector rod operable to releasably join the first clamp assembly to the second clamp assembly.

In a further embodiment an apparatus is provided to reduce bone fragments and maintain a healing position of a human's wrist and hand wherein soft tissue has been damaged. The apparatus may include a first clamp assembly operable to removably mount to an external fixator wherein the external fixator is coupled to a bone of a human, the first clamp assembly further operable to couple to a connector rod, a second clamp assembly operable to releasably couple to at least one bone pin embedded in a bone of a human and a connector rod operable to join the first clamp assembly to the second clamp assembly.

In another embodiment of the invention an apparatus for reducing bone fragments and maintaining a healing position of one or more bones of a human's wrist wherein at least one bone of the human's wrist is fractured, injured or otherwise diseased is provided. The apparatus includes a first clamp assembly operable to removably mount to an external fixator wherein the external fixator may be coupled to a radius of a human, the first clamp assembly further operable to couple to a connector rod and the first clamp assembly comprising an assembly clamp operable to attach to a longitudinal member of an external fixator, a clamp assembly head operable to secure the assembly clamp and a connector rod fastener. The apparatus may also include a second clamp assembly operable to releasably couple to a bone pin, screw or a plurality thereof embedded in the ulna of a human, the second clamp assembly comprising a releasable fastener operable to engage at least one bone pin embedded in a bone of a human's wrist, a fastener operable to hold the connector rod and a clamp assembly head and a connector rod operable to releasably join the first clamp assembly to the second clamp assembly.

In an embodiment of the invention an apparatus for reducing bone fragments and maintaining a healing position of one or more bones of a human's wrist wherein at least one bone of the human's wrist is fractured, damaged or otherwise diseased is provided. The apparatus may include a first clamp assembly operable to removably mount to an external fixator when the external fixator is coupled to a radius of a human. The first clamp assembly may be operable to couple to a connector rod. The first clamp assembly may have a generally U-shaped clamp operable to releasably attach to a longitudinal member of an external fixator. A clamp assembly head may be provided to secure the assembly clamp. A connector rod fastener may be used to releasably attach to a connector rod. The apparatus may also include a second clamp assembly operable to releasably couple to at least one bone pin embedded in the ulna of a human. The second clamp assembly may have a releasable fastener operable to engage at least one bone pin embedded in a bone of a human's wrist. A releasable rotatable fastener may be provided to hold the connector rod in position. A clamp assembly head may be used to secure the assembly clamp. The connector rod may be used to releasably join the first clamp assembly to the second clamp assembly.

In a further embodiment of the invention a method of reducing bone fragments and maintaining one or more bones of a human's wrist in a healing position is provided. The method includes attaching an external fixator to the radius bone of the wrist, releasably attaching a first clamp assembly to the external fixator, releasably attaching a second clamp assembly to at least one bone pin embedded in the ulna bone of a human and joining the first clamp assembly to the second clamp assembly with a slidable connector rod.

Technical benefits of the present invention include reducing bone fragments and maintaining a patient's hand and/or wrist in an optimum position for healing bone fractures, bone injuries or disease, and/or soft tissue damage associated with the hand or wrist. In certain types of fractures of the radius, the ulna and the distal radioulnar joint, an attachment may be added to an external fixator. Such an attachment may be desirable to further limit the range of motion of the wrist.

### BRIEF DESCRIPTION OF THE DRAWINGS

A more complete and thorough understanding of the present embodiments and advantages thereof may be acquired by referring to the following description taken in conjunction with the accompanying drawings, in which like reference numbers indicate like features, and wherein:

FIG. 1 illustrates an example of an apparatus to reduce bone fragments and maintain a healing position of bones or soft tissue of the wrist or hand;

FIG. 2 illustrates an example of an apparatus to reduce bone fragments and maintain a healing position of bones or soft tissue of the wrist or hand;

FIG. 3 illustrates an example of an apparatus to reduce bone fragments and maintain a healing position of bones or soft tissue of the wrist or hand;

FIG. 4A illustrates an example of an apparatus to reduce bone fragments and maintain a healing position of bones or soft tissue of the wrist or hand;

FIG. 4B illustrates an example of an apparatus to reduce bone fragments and maintain a healing position of bones or soft tissue of the wrist or hand;

FIG. 4C illustrates an example of an apparatus to reduce bone fragments and maintain a healing position of bones or soft tissue of the wrist or hand;

FIG. 5A illustrates an example of an apparatus to reduce bone fragments and maintain a healing position of bones or soft tissue of the wrist or hand; and

FIG. 5B illustrates an example of an apparatus to reduce bone fragments and maintain a healing position of bones or soft tissue of the wrist or hand.

### DETAILED DESCRIPTION OF THE INVENTION

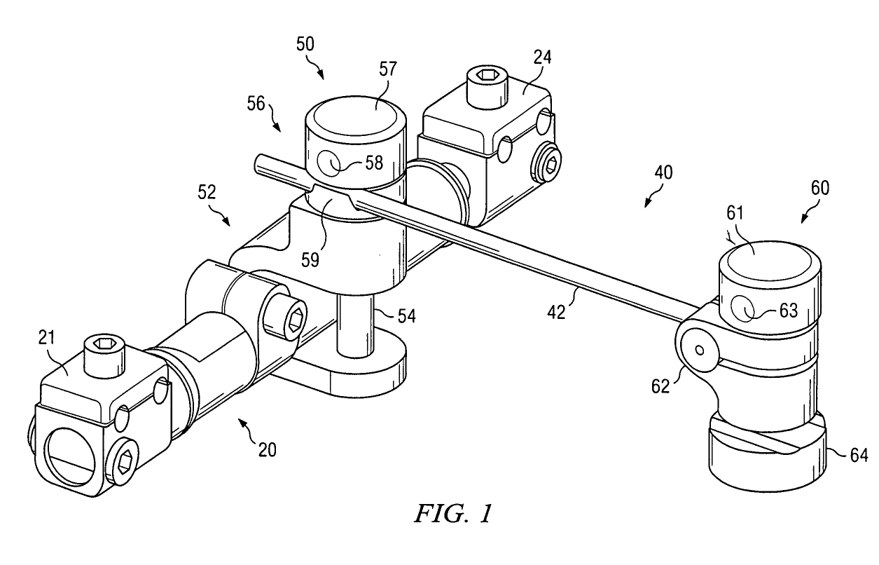

Preferred embodiments of the invention and its advantages are best understood by reference to FIGS. 1-5B, which illustrate an apparatus for reducing bone fragments and maintaining one or more bones of the wrist and hand in a healing position. The position of the apparatus on the wrist and hand may be adjusted to limit supination and pronation or other movements of the wrist, arm and hand in order to stabilize fractured bone fragments or diseased, damaged or injured bones, damaged soft tissue and promote healing of bone, soft tissue or other structures. Flexibility of pin placement and orientation of the wrist and hand and reduction of bone fragments are also features of the invention. The term “bone pin” is used in this application to include a pin, screw, rod or other mechanical device which may be engaged with a bone for attachment of a medical device.

FIG. 1 shows apparatus 40 for reducing bone fragments and maintaining one or more bones of a human's wrist and hand in a healing position, attached to external fixation device 20. External fixation device 20 may be used to immobilize some aspects of movement of a wrist and hand after injury and to further reduce bone fragments after fracture or other injury. Apparatus 40 may attach to external fixation device 20 in order to further limit movement of the wrist and hand after injury or disease, for example, after fracture of the bones of the wrist and hand. Apparatus 40, when in a therapeutic position, extends from the radial aspect of the hand and wrist to the ulnar aspect of the hand and wrist or anywhere along the ulnar aspect of the upper extremity. In this position, apparatus 40 may limit supination and pronation of the wrist, arm or hand and thereby promote healing and reduction of bony fragments. Supination is the act of turning the palm or the hand anteriorly or upward. Pronation is the act of turning the hand so that the palm faces posteriorly or downward. Standard external fixation devices alone may not have the capability to limit supination or pronation. Apparatus 40 may be affixed to external fixator 20 for the purpose of limiting any motion of the wrist, hand or arm as desired and to promote healing and reduction of bony fragments.

First clamp assembly 50 is one component of apparatus 40. First clamp assembly 50 may include bracket 52 that may releasably engage external fixator 20 or any other suitable external fixator. Clamp assembly bolt 54 may be used to secure bracket 52 to external fixator 20. Connector rod fastener 56 that forms a first attachment point for connecting rod 42 may also be coupled with clamp assembly bolt 54. Clamp assembly bolt 54 may extend from the base of bracket 52 up through clamp assembly head 57 (not expressly shown) and be locked into position by set screw or locking mechanism 58. Bracket 52 may be U-shaped or C-shaped as shown in FIG. 1 or may be any other shape or configuration suitable to attach to an external fixator. Connector rod fastener 56 may be of the type shown in FIG. 1 in which clamp assembly head 57 may be screwed down over fastener base 59 or may be any other suitable type of fastener to hold connector rod 42 or another connecting member of apparatus 40. Clamp assembly head 57 of first clamp assembly 50 may include locking mechanism 58 that maintains clamp assembly head 57 in position and keeps it from becoming loose or malpositioned. Locking mechanism 58 may be an inset screw that may be adjusted with a wrench (not expressly shown)or may be any other suitable locking mechanism that is capable of maintaining clamp assembly head 57 in position.

First clamp assembly 50 may or may not be slidable along a longitudinal axis of external fixator 20 between first or distal bone pin connector assembly 24 and second or proximal bone pin connector assembly 21. This allows the device to be used on wrists and hands of different sizes. Once a desired position along the external fixator 20 is reached, first clamp assembly 50 may be fixed into position by tightening clamp assembly bolt 54 that runs through bracket 52 of first clamp assembly 50 (not expressly shown). Clamp assembly bolt 54 may be tightened by manually turning clamp assembly head 57 and locking it into position by tightening locking mechanism 58.

First clamp assembly 50 preferably includes fastener 56 disposed on bolt 54 between clamp assembly head 57 and base 59. For some applications fastener 56 may have a generally circular configuration corresponding with clamp assembly head 57. However, a wide variety of other fasteners may be satisfactorily used with a clamp assembly incorporating teachings of the present invention. Fastener 56 may be releasably engaged with connector rod 42 having a cylindrical, square or any other suitable configuration. Connector rod 42 is preferably sized to extend across a patient's extremity, in a radial to ulnar direction, where connector rod 42 releasably couples with ulna clamp assembly 60. Ulna clamp assembly 60 may include rotatable fastener 62 that attaches to connector rod 42. Rotatable fastener 62 allows ulna clamp assembly 60 to move in superior and inferior directions as apparatus 40 is positioned on and across the wrist and hand. Ulna clamp assembly head 61 of ulna clamp assembly 60 may include locking mechanism 63 that may accept a wrench (not expressly shown) for tightening ulna clamp assembly 60.

Ulna pin clamp 64 may be configured to engage a bone pin or screw or a plurality thereof protruding from the ulna or any other bone on the ulnar aspect of the wrist, hand or arm. Ulna pin clamp 64 may engage a bone pin or screw protruding from the proximal or distal ulna, a metacarpal bone of the hand or any other suitable bone. Ulna pin clamp 64 may be configured to slide over bone pin or screw 66. Once ulna pin clamp 64 is in a desired position adjacent bone pins or screws, ulna pin clamp 64 is tightened in place by manually turning ulna clamp assembly head 61 and tightening locking mechanism 63.

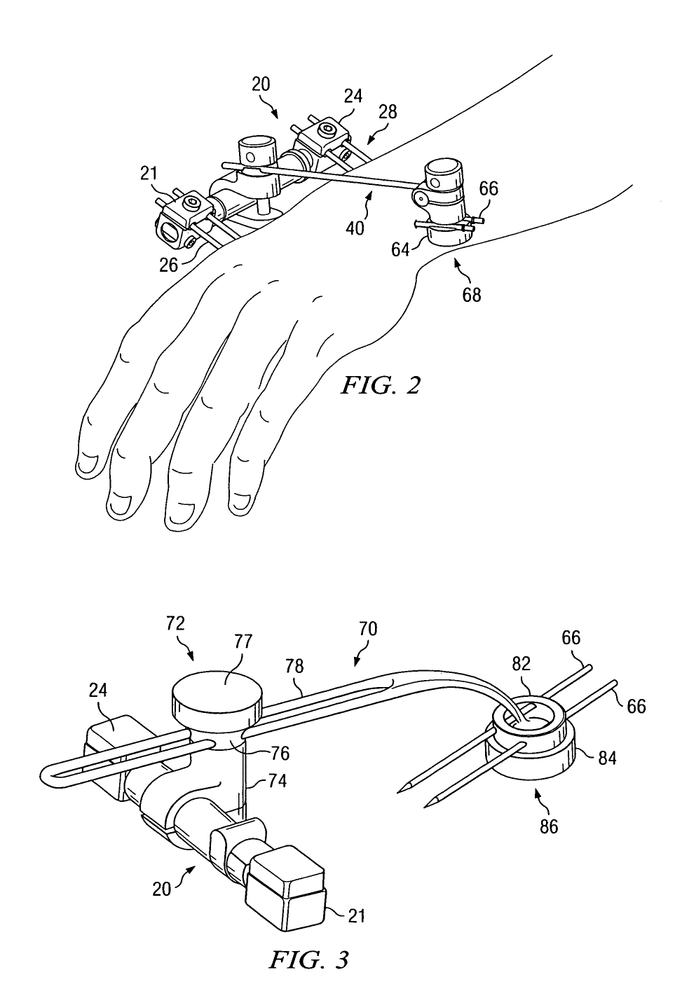

FIG. 2 shows an embodiment of external fixator 20 and apparatus 40 positioned across the wrist and hand of an upper extremity as would be required for the treatment of a fracture of the distal ulna and/or radius or other bones of the wrist and hand. External fixator 20 may be attached to the radial side of the wrist 25. In this example, first bone connector assembly 21 of external fixator 20 engages bone pins embedded in metacarpal bone 26 of a hand at a point distal to a fracture site. Second bone connector assembly 24 engages bone pins 28 embedded in the radius at a point proximal to the fracture site. Bone pins 66 placed in ulna 68 are engaged by ulna clamp 64 of ulna pin clamp assembly 60.

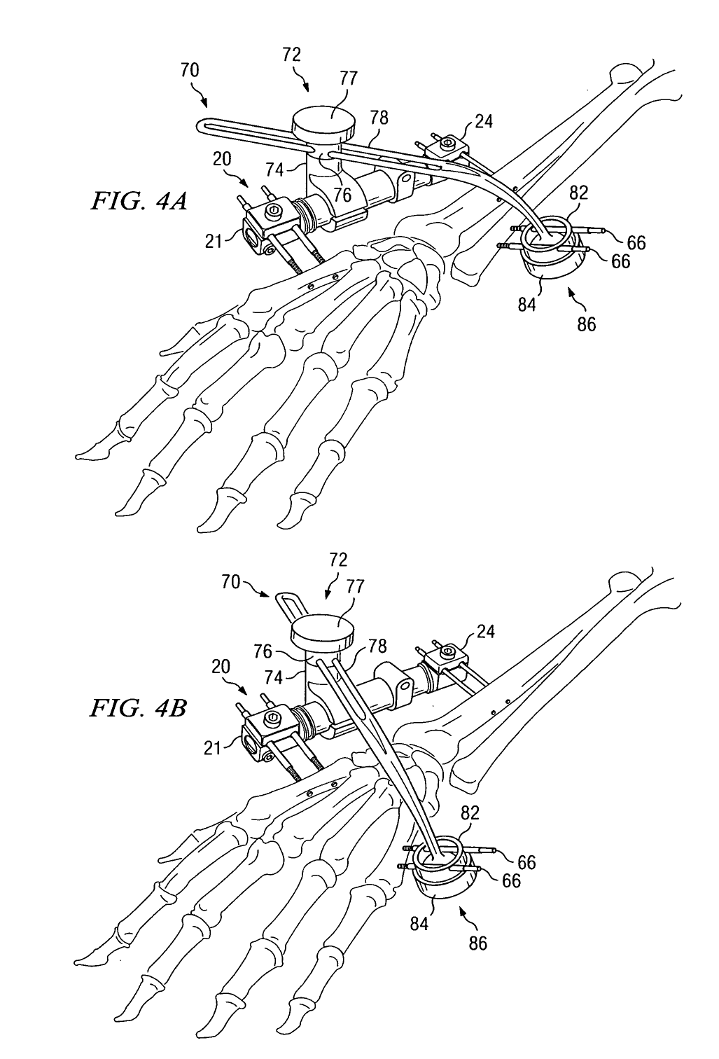

FIG. 3 shows another embodiment of apparatus 70 for reducing bony fragments and maintaining one or more bones of a human's wrist and hand in a healing position whereby first clamp assembly 72 may include clamp assembly head 77, C-shaped bracket 74, clamp assembly bolt 75, and connector rod fastener 76. First clamp assembly 72 may or may not be slidable along the longitudinal axis of external fixator 20. Once a desired position is obtained, clamp assembly bolt (not expressly shown) may be tightened to hold first clamp assembly 72 in position. Clamp assembly 72 may or may not include a locking mechanism. Connector rod 78 may include a double prong longitudinal extension that couples to connector rod fastener 76. Connector rod 78 is preferably slidable through connector rod fastener 76. When the desired position is obtained, connector rod 78 may be secured into position by tightening head 77 of first clamp assembly 72. Connector rod 78 tapers into a single prong that enlarges at its distal end into a protuberance that engages ulna clamp assembly 86. Ulna pin clamp 82 may include one or more pairs of holes such that it may be threaded onto bone pins 66 protruding from the ulna or any other suitable bone on the ulnar aspect of the wrist, arm or hand. Once ulna pin clamp 82 is positioned over ulna bone pins 66 it can be tightened by screwing down ulna pin clamp tightening mechanism 84.

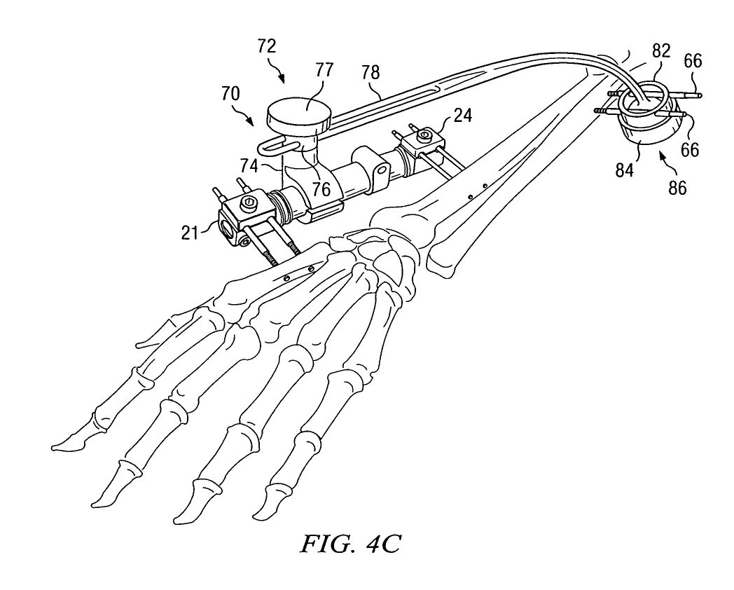

FIG. 4A shows apparatus 70 positioned on the bones of the hand and wrist. In this example, first bone connector assembly 21 of external fixator 20 is attached to bone pins 26 embedded in a metacarpal bone of a hand. Second bone connector assembly 24 of external fixator 20 is attached to bone pins embedded in the radius. First clamp assembly 72 of apparatus 70 is attached to external fixator 20 with bracket 74. Bracket 74 is tightened into position with clamp assembly bolt (not expressly shown). Bracket 74 may have a generally “C” shaped configuration or any other suitable configuration for attachment with external fixator 20. Connector rod 78 of apparatus 70 is connected to first clamp assembly 72 by connector rod fastener 76. Connector rod fastener 76 is tightened into position by manually turning clamp assembly head 77.

FIG. 4B shows an example of apparatus 70 where ulna pin clamp assembly 86 is attached to bone pins 66 embedded in a metacarpal bone of a hand. FIG. 4C shows an example of apparatus 70 where ulna pin clamp assembly 86 is attached to bone pins 66 embedded in the proximal ulna.

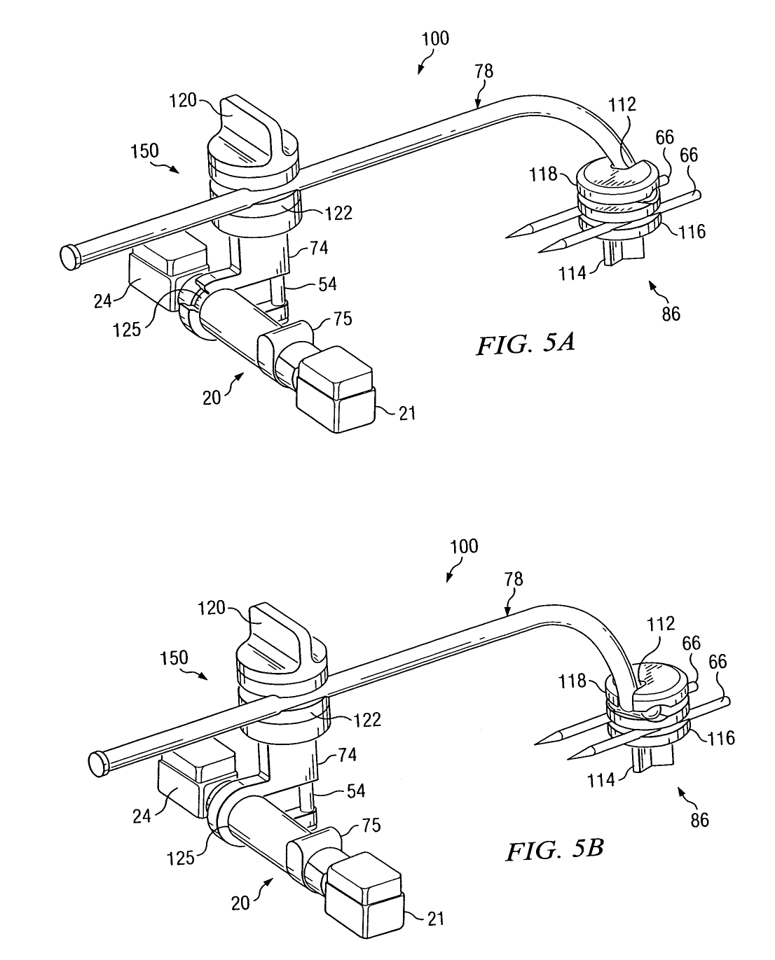

FIG. 5A shows an example of apparatus 100 for maintaining bones in a healing position. Apparatus 100 includes first clamp assembly 150 and second clamp assembly 86. First clamp assembly 150 includes bracket 74 that may be releasably engaged with external fixator 20. Clamp assembly bolt 54 may be used to secure bracket 74 to an external fixator. First clamp assembly head 120 and first clamp assembly base 122 that form a first attachment point for connecting rod 100 may also be coupled with bolt 54. Bracket 74 may be U-shaped as shown in FIG. 5A or may be any other shape, thickness or configuration suitable to attach to an external fixator. Bracket 74 may be configured to seat within groove 125 formed on external fixator 20. First clamp assembly head 120 may be screwed or clamped down on first clamp assembly base 122 to fix the position of connector rod 100. First clamp assembly 150 allows multiple degrees of freedom of movement of apparatus 100 permitting variation in positioning of apparatus 100 depending on the angle of fixation and/or reduction required. First clamp assembly head 120 may include a locking mechanism such as a set screw (not expressly shown). First clamp assembly 150 may be slidable along a longitudinal axis of external fixator 20 between first or distal bone pin connector assembly 21 and second or proximal bone pin connector assembly 24.

Connector rod 78 is positioned to extend across a human's extremity, in a radial to ulnar direction, where connector rod 78 releasably couples with ulna clamp assembly 86. Ulna clamp assembly 86 may include a rotatable fastener or hinge 112 that attaches connector rod 78 to ulna clamp assembly 86 and permits varying degrees of angulation of ulna clamp assembly 86. Ulna clamp assembly head 118 of ulna clamp assembly 86 may include a locking mechanism (not expressly shown) for tightening ulna clamp assembly 86 into position. Ulna clamp assembly 86 attaches to bone pins 66 that may be embedded anywhere along the ulnar aspect of an extremity from the proximal ulna at the elbow to the distal ulna at the wrist or in one or more metacarpal bones of the hand. Bone pins 66 may be positioned between ulna clamp assembly head 118 and ulna clamp assembly base 116. Ulna clamp assembly may include tightening knob or bolt 114 used to tighten ulna clamp assembly 86 around bone pins 66.

FIG. 5B shows apparatus 100 similar to 5A with rotatable fastener or hinge 112 in an alternate position that may be desirable in some circumstances to allow an alternate pivot point for ulna clamp assembly 86.

Although the present invention and its advantages have been described in detail, it should be understood that various changes, substitutions and alternations can be made herein without departing from the spirit and scope of the invention as defined by the following claims.

What is claimed is:

1. An apparatus for and maintaining a wrist in a healing position comprising:
a first clamp assembly operable to removably mount to an external fixator;
the first clamp assembly operable to couple to a connector rod;
a second clamp assembly operable to releasably couple to at least one bone pin embedded in a bone; and
the connector rod operable to join the first clamp assembly to the second clamp assembly.

2. The apparatus of claim 1 further comprising the external fixator attached to a human's radius and the second clamp assembly coupled to a bone pin embedded in a human's ulna.

3. The apparatus of claim 1 further comprising the external fixator attached to a human's radius and the second clamp assembly coupled to a bone pin embedded in at least one metacarpal bone of a human.

4. The apparatus of claim 1 further comprising the apparatus formed of at least one of a group consisting of stainless steel, titanium, plastic and radiolucent material.

5. The apparatus of claim 1 further comprising the first clamp assembly and the second clamp assembly operable to maintain a human's radius and ulna bones in a neutral position.

6. The apparatus of claim 1 further comprising the first clamp assembly and the second clamp assembly operable to maintain a human's radius and ulna bones in a supinated position.

7. The apparatus of claim 1 further comprising the first clamp assembly, second clamp assembly and the connector rod operable to limit mobility of a human's radius and ulna bones by positioning the radius and ulna bones.

8. The apparatus of claim 1 further comprising the first clamp assembly and the second clamp assembly operable to allow supination and pronation of the radius and ulna bones to be limited by intraoperative manipulation of the apparatus.

9. The apparatus of claim 1 wherein the first clamp assembly comprises an assembly clamp operable to attach to a longitudinal member of an external fixator and a clamp assembly head operable to secure the assembly clamp and a connector rod fastener.

10. The apparatus of claim 1 wherein the second clamp assembly comprises:
a fastener operable to releasably engage at least one bone pin embedded in a bone of a human's arm; and
the fastener operable to releasably hold the connector rod and a clamp assembly head.

11. The apparatus of claim 1 wherein the second clamp assembly comprises:
a fastener operable to releasably engage at least one bone pin embedded in a bone of a human's hand; and
the fastener operable to releasably hold the connector rod and a clamp assembly head.

12. The apparatus of claim 1 wherein the apparatus is positioned to reduce bony fragments and maintain the bony fragments in position.

13. An apparatus for maintaining a healing position of one or more bones of a human's arm comprising:
a first clamp assembly operable to removably mount to an external fixator coupled to a radius of a human;
the first clamp assembly further operable to couple to a connector rod;
a second clamp assembly operable to releasably couple to at least one bone pin embedded in a bone of a human; and
a connector rod operable to releasably join the first clamp assembly to the second clamp assembly.

14. The apparatus of claim 13 wherein the first clamp assembly comprises a U-shaped bracket operable to attach to a rod-shaped component of an external fixator and the first clamp assembly further comprising a fastener operable to couple to the connector rod.

15. The apparatus of claim 13 wherein the second clamp assembly comprises a rotatable fastener configured to couple the second clamp assembly to the connector rod and the second clamp assembly further comprising a releasable clamp operable to engage a bone pin or screw embedded in a human's bone.

16. The apparatus of claim 13 wherein the first clamp assembly comprises a clamp operable to attach to a longitudinal member of an external fixator and a clamp assembly head operable to secure the assembly clamp and a connector rod fastener.

17. The apparatus of claim 13 wherein the second clamp assembly comprises a releasable fastener operable to engage at least one bone pin or screw embedded in a bone of a human's arm and a fastener operable to hold the connector rod and a clamp assembly head.

18. The apparatus of claim 13 further comprising the at least one bone pin embedded in a bone selected from a group consisting of the radius, ulna and at least one metacarpal bone.

19. An apparatus for maintaining a healing position of one or more bones of a human's wrist wherein at least one bone of the human's wrist is injured comprising:
a first clamp assembly operable to removably mount to an external fixator when the external fixator is coupled to a bone of a human;
the first clamp assembly further operable to couple to a connector rod;
a second clamp assembly operable to releasably couple to at least one bone pin embedded in a bone of a human; and
a connector rod operable to join the first clamp assembly to the second clamp assembly.

20. The apparatus of claim 19 wherein the first clamp assembly comprises:
a bracket operable to attach to a longitudinal member of an external fixator; and
a clamp assembly head operable to secure the assembly clamp and a connector rod fastener with the bracket.

21. The apparatus of claim 19 wherein the second clamp assembly comprises:
a releasable fastener operable to engage at least one bone pin or screw embedded in a bone of a human's wrist; and
a fastener operable to hold the connector rod and a clamp assembly head.

22. The apparatus of claim 19 further comprising the external fixator attached to a radius bone of a human.

23. The apparatus of claim 19 further comprising the external fixator is attached to at least one metacarpal bone of a human.

24. The apparatus of claim 19 wherein the connector rod comprises a single longitudinal member.

25. The apparatus of claim 19 wherein the connector rod comprises a first end with at least one prong operable to slidably attach to the first assembly clamp and a second end having a body operable to engage the second assembly clamp.

26. The second assembly clamp of claim 19 further comprising:
a circular head having a central opening; and
the central opening operable to engage the connector rod.

27. The apparatus of claim 19 wherein the apparatus is positioned to reduce bony fragments.

28. The apparatus of claim 19 wherein the apparatus is positioned to maintain position of injured soft tissue.

29. An apparatus for reducing bony fragments and maintaining a healing position for at least one bone or soft tissue of a human's wrist or hand comprising:
a first clamp assembly operable to removably mount to an external fixator wherein the external fixator is coupled to a radius of a human, the first clamp assembly further operable to couple to a connector rod and the first clamp assembly comprising an assembly clamp operable to attach to a longitudinal member of an external fixator, a clamp assembly head operable to secure the assembly clamp and a connector rod fastener;
a second clamp assembly operable to releasably couple to a bone pin, screw or a plurality thereof embedded in the bone of a human, the second clamp assembly comprising a releasable fastener operable to engage at least one bone pin embedded in a bone of a human, a fastener operable to hold the connector rod and a clamp assembly head; and
a connector rod operable to releasably join the first clamp assembly to the second clamp assembly.

30. An apparatus for maintaining a healing position of one or more bones of a human's wrist wherein at least one bone of the human's wrist requires fixation comprising:
a first clamp assembly operable to removably mount to an external fixator wherein the external fixator is coupled to a radius of a human, the first clamp assembly further operable to couple to a connector rod and the first clamp assembly comprising a U-shaped clamp operable to releasably attach to a longitudinal member of an external fixator, a clamp assembly head operable to secure the assembly clamp and a connector rod fastener operable to releasably attach to a connector rod;
a second clamp assembly operable to releasably couple to at least one bone pin embedded in the bone of a human, the second clamp assembly comprising a releasable fastener operable to engage at least one bone pin embedded in a bone of a human, a releasable rotatable fastener operable to hold the connector rod in position and a clamp assembly head operable to secure the assembly clamp; and
the connector rod operable to releasably join the first clamp assembly to the second clamp assembly.

31. A method of maintaining a human's wrist or hand in a healing position comprising:
attaching an external fixator to the radius bone of a wrist;
releasably attaching a first clamp assembly to the external fixator;
releasably attaching a second clamp assembly to at least one bone pin embedded in a bone of a human; and
joining the first clamp assembly to the second clamp assembly with a connector rod.

32. The method of claim 31 further comprising limiting the degree of supination and pronation of a human's radius and ulna bones by positioning the first clamp assembly, second clamp assembly and connector rod relative to the human's radius and ulna bones.

33. The method of claim 31 further comprising reducing bony fragments of an extremity.

34. A method of maintaining a human's wrist or hand in a healing position comprising:
limiting the degree of supination and pronation of the human's radius and ulna bones as required for healing of an injury to the human's wrist or hand.

35. The method of claim 34 further comprising attaching an external fixator to the radius bone of the wrist;
releasably attaching a first clamp assembly to the external fixator;
releasably attaching a second clamp assembly to at least one bone pin embedded in a bone of a human;
joining the first clamp assembly to the second clamp assembly with a connector rod;
positioning the first clamp assembly, the second clamp assembly and the connector rod relative to the radius bone and the ulna bone to maintain the desired healing position.

36. A method of reducing bony fragments in a human's wrist or hand comprising:
attaching an external fixator to the radius bone of a wrist;
releasably attaching a first clamp assembly to the external fixator;
releasably attaching a second clamp assembly to at least one bone pin embedded in a bone of a human; and
joining the first clamp assembly to the second clamp assembly with a connector rod.
